(19) United States
(12) Patent Application Publication
(10) Pub. No.: US 2011/0027827 A1
(43) Pub. Date: Feb. 3, 2011

(54) INTEGRATED SYSTEM FOR PRODUCTION OF BIOFUEL FEEDSTOCK

(76) Inventors: Zhanyou Chi, Pullman, WA (US); Yubin Zheng, Pullman, WA (US); Ben Lucker, Moscow, ID (US); Shulin Chen, Pullman, WA (US)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 12/512,337

(22) Filed: Jul. 30, 2009

(51) Int. Cl. C12P 1/00; C12M 1/00; C12M 1/34; C12M 1/38

(52) U.S. Cl. 435/41; 435/292.1; 435/287.1; 435/286.1

(57) ABSTRACT

Disclosed is a culture system for the production of algae biomass to obtain lipid, protein and carbohydrate. By integrating heterotrophic processes with a phototrophic process in parallel, this system provides year around production in colder climates. By integrating heterotrophic processes with a phototrophic process in series, this system creates a two-stage, separated mixed-trophic algal process that uses organic carbon and nutrients for the production of seed in the heterotrophic process, followed by release of cultured seed in large-scale phototrophic culture for cell biomass accumulation. Organic carbon source including waste materials can be used to feed the heterotrophic process. The production capacity ratio between the heterotrophic and the phototrophic processes can be adjusted according to season and according to the availability of related resources. The systems are used for producing and harvesting an algal biofuel feedstock as well as other potential high-value products. The sequence and approach enhances utilization of carbon and nutrient waste-streams, provides an effective method for controlling contamination, adds flexibility in regard to production and type of available products, and supplies greater economic viability due to maximized use of available growth surface areas.

### CROSS REFERENCE TO RELATED APPLICATIONS

This application claims benefit of U.S. provisional patent application Ser. No. 60/084,708 filed Jul. 30, 2008, the complete contents of which are hereby incorporated by reference.

### FIELD OF THE INVENTION

Disclosed is a culture system for the production of algae biomass to obtain lipid, protein and carbohydrate. By integrating heterotrophic processes with phototrophic processes in parallel, this system provides year around production in colder climates. By integrating heterotrophic process with phototrophic process in series, this system creates a two-stage, separated mixed-trophic algal process that uses organic carbon and nutrients for the production of seed in the heterotrophic process, followed by release of the cultured seed in large-scale phototrophic culture for cell biomass accumulation. Organic carbon sources including waste materials can be used to feed the heterotrophic process. The production capacity ratio between the heterotrophic and the phototrophic processes can be adjusted according to season and according to the availability of related resources. The systems are used for producing and harvesting an algal biofuel feedstock as well as other potential high-value products. The sequence and approach enhances utilization of carbon and nutrient waste-streams, provides an effective method for controlling contamination, adds flexibility in regard to production and type of available products, and supplies greater economic viability due to maximized use of available growth surface areas.

### BACKGROUND

Lipid production using microorganisms such as algae and yeast have the potential to produce much higher amounts of oil per unit land area than oil seed crops (Pienkos and Darzins, 2009). It has been suggested that algae have the possibility to displace a significant amount of fossil fuel without causing great land use change or impact. According to Chisti (Chisti, 2007), the US would only need approximately 1.1% and 2.5% of existing cropping area to meet 50% of all transportation fuel needs of the United States assuming 70% and 30% of oil in dry algae biomass, respectively. If used as a means for CO₂ sequestration, 1.6-1.8 lb of CO₂ will be fixed for each pound of algae biomass produced (Chisti, 2007). In addition, algae culture as a form of aquaculture provides new opportunities for agriculture, leading to economic benefits for producers, processers, and distributors. Clearly, new technologies related to algae production as a feedstock for biofuel production offer great opportunities in meeting society's needs for energy security, combating climate change, and sustainable economic development.

Algae culture can be divided into three types according to the source of carbon that is used as the energy carrier as well as the building block of the algae cells. The first type is phototrophic culture in which carbon dioxide is used as the carbon source and sunlight is used as the energy source. The second type is heterotrophic culture in which organic carbon such as glucose or organic acids is used as both the carbon and energy source. The third type is mixed-trophic culture in which both carbon dioxide and organic carbon are used as carbon sources. Presently, none of these culture processes is used at a commercial level for fuel production due to the lack of enabling technologies, as technical barriers exist in each of the technologies.

Phototrophic algae culture, comparatively, has a very low productivity (Singh and Ward, 1997) (Wen and Chen, 2003) as well as concerns regarding scale and capital cost. Moreover, light limitation cannot be entirely overcome since light penetration is inversely proportional to the cell concentration. The high concentration of oxygen accumulation in the culture of photo-bioreactors is another unsolved problem. Phototrophic algae culture in open ponds takes advantage of low operating costs, but the productivity is too low, and it is easily contaminated by invading species and insects (Rusch and Christensen, 1998; Rusch and Malone, 1998). Economically separating the low density biomass from a huge volume of water is another hurdle (Knuckey et al., 2006). So far, the phototrophic algae are only commercially used to produce high value products such as pigments, carotenoids and nutraceuticals. Large scale culture of phototrophic algae for biodiesel production still has too high a production cost, compared to the produced value (Pienkos and Darzins, 2009)

Unlike phototrophic algae, heterotrophic oleaginous microorganisms such as algae or yeast do not need light, thus can be cultured at higher density in larger vessels. Heterotrophic culture also takes advantage of fast growth kinetics, high production, and easy harvest. The early exploration of single cell oil (SCO) production by yeast fermentation started in the 1980s, with attempts to produce commercial scale of SCO, as well as single cell protein (SCP), from microorganisms to substitute the lipids and proteins from plants as a source of food for human and feed for animals (Ward and Singh, 2005). Early SCO production focused on high value oils such as cocoa butter, and some fermentation processes with yeast were later commercialized. From the 1990s, a series of heterotrophic microalgae species were used to produce high value polyunsaturated fatty acids (PUFA) such as omega-3 fatty acids to substitute the traditional source of fish oil. Some of them, such as species from Schizochytrium sp. and Cryptocodinium cohnii were successfully used in industry-scale DHA production processes (Bailey et al., 2003) and used as supplements for infant formula. Recently, some research on yeast and heterotrophic microalgae culture to produce biodiesel was reported and showed the promise of this process (Easterling et al., 2009; Xiong et al., 2008; Xue et al., 2008). However, the hurdles for heterotrophic microorganisms for biodiesel production come from the high cost of the culturing process and the high cost of carbon sources as feedstocks. The conversion rate from sugar to lipid ranges from about 16.7% to 25%, depending on the lipid content in the produced yeast or algae biomass, since a certain amount of carbon has to be used to provide energy and some in other forms to form cell structure and cell mass (Kyle and Ratledge, 1992). Thus, it takes about 4-6 tons of glucose to produce 1 ton of oil. Utilization of cellulosic-derived sugars as a means to reduce feedstock cost is another option but is still too costly with technical hurdles still remaining, as evidenced by delays in developing and commercializing cellulosic ethanol production. An alternative low cost solution is through utilization of waste organic resources rich in sugars and/or starches as sources for organic carbon in the heterotrophic process (Kapdan and Kargi, 2006). Although limited in availability, this type of organic waste is sufficient and ideal for use as a carbon resource in heterotrophic seed reactors while also supplying valuable micro and macro nutrients to not only the seed reactors but to downstream phototrophic ponds, both of which are at the core of the growth innovation described below.

### SUMMARY OF THE INVENTION

This invention takes the best of high-cell density heterotrophic production and the benefits of low processing costs of large-scale phototrophic growth and integrates them in such a way as to optimally recycle and utilize waste carbon and nutrients while also utilizing waste organic feedstocks. This is done all in a manner which enhances flexibility, overcomes seasonal weather variations in cold climates, reduces concerns regarding contamination, and lowers required pond size and associated capital cost. First, the system, if need be, can operate the heterotrophic and phototrophic culture processes in parallel allowing the use of both organic and inorganic carbon sources at a given site to expand the capacity of the production and the stability of the system, especially against inclement seasonal weather and/or phototrophic contamination. In that case, the phototrophic algae culture process will be shut down, but the heterotrophic process such as oleaginous yeast and heterotrophic algae culture using organic waste as feedstock will be run alone to produce oil-enriched yeast or algae biomass, which then can be processed into biofuel.

Most importantly, the system also integrates heterotrophic with phototrophic culture processes in series to grow mixed-trophic algae, creating a separated mixed-trophic culture process in which heterotrophic culture for seed production is followed by a phototrophic one for biomass and lipid accumulation. The in-series process is enabled through the use of lipid-yielding phototrophic algae which are also facultative heterotrophs. More specifically many Chlorella sp (Hermsmeier et al., 1991), Chlamydomonas sp. (Boyle and Morgan, 2009), Scenedesmus sp. (Abeliovich and Weisman, 1978), and many species of diatoms are capable of duel trophism (Lewin, 1953). This ability of dual trophism can be taken advantage of for the industrial use of algae cultivation for biomass production. As discussed in detail below, by taking advantage of this dual trophism, the entire integrated system gains numerous competitive advantages, a non-exhaustive list thereof being presented below.
1) Integrating a heterotrophic component (module) in parallel with a phototrophic production process to ensure production capacity in colder seasons and to expand the production capacity at a selected site. For such a purpose, the heterotrophic culture system is also applicable to yeast.
2) Heterotrophic seed growth is advantageous to the normally-utilized phototrophic seed growth in that the heterotrophic process can be more efficient, producing seeds of greater inoculums concentration in a shorter period of time.
3) The heterotrophic process utilizes organic carbon and as such allows for use of organic materials from a variety of organic waste streams, even recycled waste glycerol, that are often readily available and at times received with associated tipping fees for enhanced economics. The use of organic carbon to in part complete the algal biomass development brings added flexibility and diversity to the operation, not just relying on ambient or industrial CO₂, and producing the aforementioned opportunity whereby dedicated heterotrophic reactors could be reformulated to serve in parallel or singly during periods of weather or contamination concern. Preferably, waste solids are pretreated and hydrolyzed to produce the easy-to use organic carbon to the species to be cultured. The temperature for this culture is between 10-40 C, and more preferably, 20-35 C, and most preferably 30-35 C. Oxygen will be supplied to the culture vessel used in this process, and the dissolved oxygen controlled in this process is between 0-90% of the saturated oxygen concentration in the culture broth, and more preferably 5-50% of the saturated oxygen concentration in the culture broth. The fermentor or bioreactor used in this process is one or more of the following culture vessels: stirred tank fermentor, bubble column bioreactor, air lift bioreactor, or any other bioreactor can be used to culture heterotrophic microorganisms. Herein, reactors used for heterotrophic growth may be referred to as “dark” fermentors, reactors or bioreactors.
4) Enhancing the capability of seed production with the heterotrophic process provides a way to control contaminants down-stream within the phototrophic growth process as the seed production capacity can be increased significantly if needed. Evidence clearly shows that the type, number and size of inoculums, as optimally produced by the heterotrophic process, have direct effects on an open phototrophic structure's ability to overcome contamination.
5) Throughout the sequence of growth, carbon and nutrient utilization is optimized with produced CO₂ and excess nutrients remaining within the heterotrophic spent media, being able to be utilized during phototrophic growth, ultimately leading to greater efficiency of chemical inputs and solid and wastewater management.
6) By seeding through a dedicated heterotrophic process, available surface area within the phototrophic ponds is maximized for growth productivity, thereby resulting in enhanced economics.
7) The process and utilization of multiple carbon sources, opens up viable algal biomass cultivation to climatic zones perhaps previously non-viable due to temperature and/or solar intensity, recalibrating small deficiencies in inorganic carbon utilization through the use of organic carbon available within the region. The result is a more robust and economically and environmentally viable process that more efficiently recycles and utilizes the carbon and nutrient inputs, thereby increasing the sustainability of the system.

The sequence of culture is a novel approach in that previous inventions applied to algal biomass for biofuel feedstock have relied either solely on various processes of phototrophic-alone, heterotrophic-alone (Chen and Chen, 2006), and/or a phototrophic to heterotrophic sequence, not the reverse. Specific to the latter, United States patent application 20080160593-A1 (Oyler, 2008), which is herein incorporated by reference, described a process for production of biofuels from algae via sequential photoautotrophic and heterotrophic growth. Core to the reverse application as described in this invention is the use of phototrophic algae with facultative heterotrophic ability. While studying the facultative heterotrophic capabilities of certain algal species Chen and Johns (1991) investigated the effect of C/N ratio and aeration on the fatty acid composition of heterotrophic Chlorella sorokinina, but drew no conclusions on use of the species and like-species for this application or sequence of processing. A notable attribute of the described invention and sequence of growth is the ability for the system to reduce down-stream, open-pond contamination. Previously, Theegala et al (1999) reported a strategy for contamination washout in a hydraulically integrated serial turbidostat algal reactor, thus focusing on flow rates and residence times as a means for contamination control, but not making note of the role inoculums can play for the same purpose.

The invention provides a method of producing biofuel feedstock. The method comprises the steps of: using heterotrophic culture of algae or algae like species for seed production, the algae or algae like species having physiological mechanisms for both phototrophic and heterotrophic growth; and then using seed produced from said heterotrophic culture in a phototrophic culture for biomass and lipid accumulation in the algae or algae like species. In one embodiment, the using heterotrophic culture step includes a step of using waste water and waste solids as an organic carbon source, to culture the algae or algae like species having physiological mechanisms for both phototrophic and heterotrophic growth in a dark fermenter or bioreactor. In some embodiments, the waste water and waste solids include but are not limited to agricultural wastes, industrial pulps, organic fraction municipal waste, algae biomass residue, or any other organic waste which contains available organic carbon to be utilized by the cultured microorganisms. The method may further include a step of inputting carbon dioxide produced during the using heterotrophic culture step as a carbon source into the using phototropic culture step. In some embodiments, the algae or algae like species are, for example, Chlorella sp., Chlamydomonas sp., and/or Scenedesmus sp. In some embodiments, seed produced from the heterotrophic culture has a cell count ranging from about 10⁷ to about 10⁹ cells/ml, or more. The method may further comprise a step of using the heterotrophic culture of algae or algae like species for biomass and lipid accumulation.

The invention also provides a method of producing biofuel, the method comprising the steps of: using heterotrophic culture of algae or algae like species for seed production, the algae or algae like species having physiological mechanisms for both phototrophic and heterotrophic growth; and then using seed produced from the heterotrophic culture in a phototrophic culture for biomass and lipid accumulation in the algae or algae like species; and recovering lipids from algae or algae like species culture in the phototrophic culture for use as biofuel. In one embodiment, the using heterotrophic culture step includes the step of using waste water and waste solids as an organic carbon source, to culture the algae or algae like species having physiological mechanisms for both phototrophic and heterotrophic growth in a dark fermenter or bioreactor. The waste water and waste solids include but are not limited to agricultural wastes, industrial pulps, organic fraction municipal waste, and algae biomass residue. This method may also, in some embodiments, comprise the step of inputting carbon dioxide produced during the using heterotrophic culture step as a carbon source into the using phototropic culture step. The algae or algae like species used in the practice of the method include but are not limited to Chlorella sp., Chlamydomonas sp., and Scenedesmus sp. Seed produced from the heterotrophic culture generally has a cell count ranging from about 10⁷ to about10⁹ cells/ml, or more.

The invention also provides systems for producing biofuel feedstock. The systems comprise: at least one tank for heterotrophic culture of algae or algae like species for seed production, the algae or algae like species having physiological mechanisms for both phototrophic and heterotrophic growth; and at least one open pond or reactor for phototrophic culture for biomass and lipid accumulation in the algae or algae like species using seed produced from the heterotrophic culture(s). An open pond or reactor is configured so as to receive seed algae or algae like species from at least one tank for heterotrophic culture. The system may include a detector (or multiple detectors) to detect and provide output regarding growth conditions in open ponds or reactors, and may further include one or more controllers to automatically shut down or start up operation of the open ponds or reactors in response to the output provided by the detector. Parameters (growth conditions) that may be monitored include but are not limited to the amount of light, the temperature, and the presence (or absence) of contamination.

### BRIEF DESCRIPTION OF THE DRAWINGS

FIG. 1. System overview for integrated system of heterotrophic-phototrophic algae culture

FIG. 2. Mass balance for an integrated system of heterotrophic-phototrophic algae culture

FIG. 3. Yeast culture with food waste hydrolysis

FIG. 4. Glucose supplemented culture of oleaginous yeast with municipal wastewater

FIG. 5. The growth kinetics of seed cells of C. sorokiniana from food waste culture

FIG. 6. Comparison of doubling rates for different seed growth processes

FIG. 7. The growth curve of Chlorella Sorokiniana versus EG-1-2

FIG. 8. Comparison of doubling rates in large open cultures from heterotrophic and phototrophic seed cultures

FIGS. 9a-b. Biomass growth comparison between heterotrophic and phototrophic seeding regimes followed by phototrophic growth (same inoculums size) (FIG. 9a) and engineering cost comparisons (FIG. 9b)

FIG. 10. Economic comparison of mixed-trophic and phototrophic-alone systems

### DETAILED DESCRIPTION OF THE INVENTION

Herein a two-stage algal growth process wherein the product of an initial high-cell density, heterotrophic growth stage provides inoculums for a subsequent phototrophic growth stage is described. The growth process is suited for use with algal species that have physiological mechanisms capable of both phototrophic and heterotrophic growth. More specifically many Chlorella sp., Chlamydomonas sp., Scenedesmus sp., and many species of diatoms are capable of duel trophism. This ability of duel trophism can be taken advantage of for the industrial use of algae cultivation for biomass production or accumulation.

By “biomass production” or “biomass accumulation” we mean an increase in the total number of organisms that are present in a culture over time and/or an increase in the amount of particular substances that are produced or synthesized by individual organisms during a period of time in culture. In other words, “biomass” may refer to the components of the organisms themselves, or to substances that are produced by the organisms. Such substances may accumulate within the organisms, or may be part of the organisms, or be attached to the organisms, or may be secreted into the culture medium by the organisms. Examples of substances that are so-produced include but are not limited to, for example, lipids, proteins, carbohydrates, sugars, amino acids, carotenoids, etc. Such substances may be produced constitutively by the organisms throughout growth, and the amount of the substance in the culture increase simply due to an increase in the number of organisms. Alternatively, the production of such substances may be induced in response to culture conditions or other environmental cues (e.g. nitrogen starvation). In particular, lipid components of the biomass may be used as biofuel.

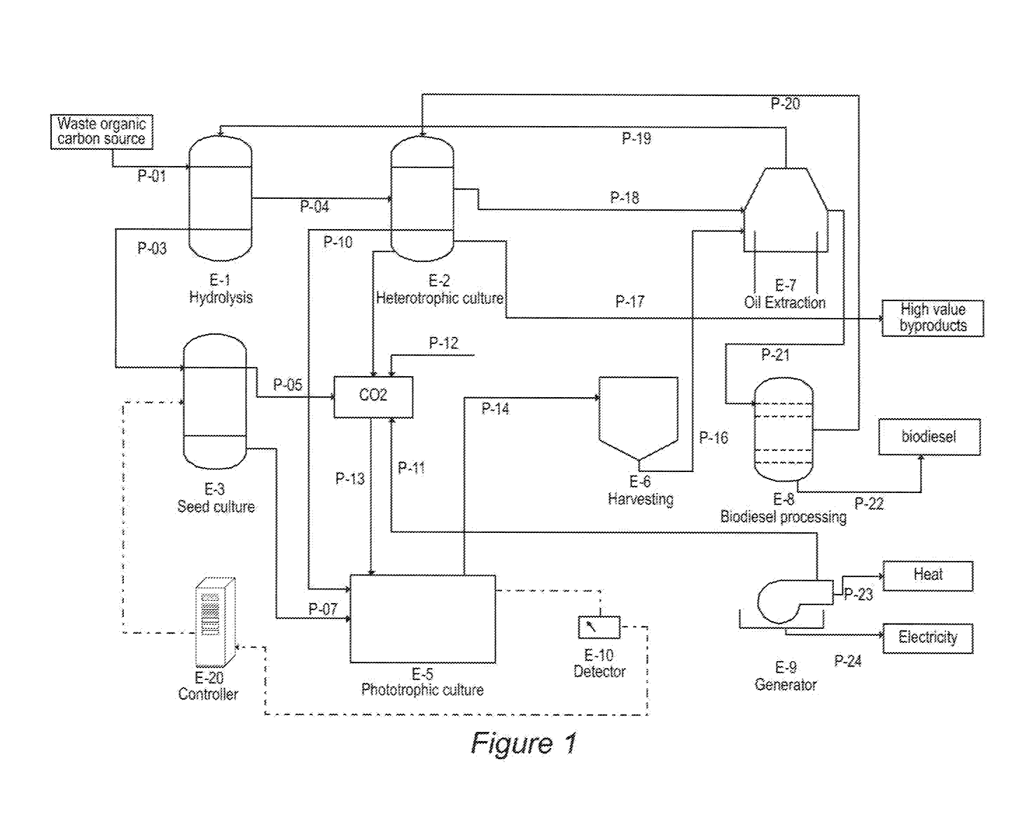

In a preferred embodiment waste carbon sources are utilized as a source of nutrient for the first, heterotrophic growth stage. The product of the first growth stage serves as an inoculum for the second growth stage. Upon completion of the first stage of growth, there remains, upon dilution, sufficient nutrient for the second, phototrophic stage as well as available CO₂ that can be recovered and utilized (FIG. 1).

In the practice of the invention, one or more organic carbon sources are provided to support growth of the heterotrophic culture. In one embodiment of the invention, the organic carbon source is waste material. The waste material may be or may be derived from waste water and/or solid waste products that contain organic carbons sources that can be utilized by the heterotrophic organisms. This embodiment is advantageous in terms of cost savings with respect to supplies, and in terms of helping to solve problems of waste disposal. Waste organics (such as agricultural wastes, industrial pulps, organic fraction municipal waste, algae biomass residue, various waste waters, etc.) are first received, preferably in a tipping fee arrangement, at the facility (P-01, FIG. 1) and hydrolyzed, if necessary, for sugar feedstock production (E1). In some embodiments, to hydrolyze the waste organics, a protocol of dilute acid pre-hydrolysis followed by enzymatic hydrolysis will be used. Dilute acid pretreatment with sulfuric acid is considered the most cost effective means of hydrolyzing wood and agricultural residues by breaking down the hemicellulose structure, reducing the cellulose crystallinity and increasing the porosity for subsequent enzymatic action. Under acidic conditions some proteins can be simultaneously hydrolyzed into amino acids for utilization by algae and yeast. After acid pretreatment the enzymatic hydrolysis process using enzymes such as cellulases and hemicellulases can be used to produce sugars. Recycled crude glycerol and spent biomass from a downstream esterification process (E-7) can also serve as a carbon and nutrient feed (P-20 and P-19). Sugars and/or glycerol can then be sent (P-03 and P-04) to either of two heterotrophic processing tanks (E2 and E3)—one for obligate heterotrophic growth for value-added products (P-17) and another for heterotrophic growth wherein the product is a seed culture (P-07) for a second stage phototrophic open pond (E-5). In addition, such carbon sources can be supplemented with non-waste carbon sources, and/or with non-waste water, if needed.

Other than biofuel, potential value-added products from the obligate heterotrophic algae include carotenoids, enzymes, and in a preferred embodiment, DHA and other omega-3 fatty acids. This obligate heterotrophic growth utilizes processes similar to that which are described in U.S. patent application Ser. No. 12/132,131, which is herein incorporated by reference. The value-added products contained in the heterotrophic algae can be utilized and marketed directly as an algal feed or can be further processed for downstream separation. In a preferred embodiment of the downstream separation, the algal biomass (P-18) is directly converted to mixed fatty acid methyl esters (FAME) (P-21) and other by-products (P-19) using an esterification process such as that described in PCT/US08/50799, which is herein incorporated by reference, that requires no oil extraction or drying (E-7). In particular embodiments wherein the value-added product comprises an omega-3 fatty acid, the resulting mixed FAME (P-21) can then be further separated using distillation (E-8) into a nutraceutical omega-3 FAME and a non-omega-3 FAME that can be sold as biodiesel (P-22).

Most phototrophic algae are also facultative heterotrophs in that they have the required mechanisms and therefore the capability to utilize and convert organic carbon. In this process phototrophic algae which are also facultative heterotrophs are cultivated in a two-stage process. The first stage is a closed heterotrophic growth or seed-cell process which serves to generate a high cell count (e.g. from about 10⁷ cells/ml to about 10⁹ cells/ml or more) utilizing processes similar to those described in U.S. patent application Ser. No. 12/132,131, which is herein incorporated by reference. The nutrient and cell dense product of this first stage is utilized to seed and fertilize the second stage: phototrophic open pond or photobioreactor growth. This two-stage process allows for greater control of potential open pond contamination through the use of high-cell density inoculants that are produced in the first stage of growth. The first stage of growth provides cells that have the number (from about 10⁷ cells/ml to about 10⁹ cells/ml, or more) to effectively seed as well as survive in a second-stage open-air phototrophic process. In many situations seed cultures for algae are generated in photo-bioreactors and or in open ponds. However, both of these processes require illuminated surface area, which either is very expensive and does not economically scale (enclosed photo-bioreactors) or competes with high density cultures for sunlight illuminated surface area. Heterotrophic fermentors do not require high surface area to volume ratios for growth as phototrophic growth processes do, and therefore are more easily scaled and do not compete with illuminated surface area of the dense open cultures. Another advantage of heterotrophic seed cultures is the ability to control chlorophyll content of the seed cells through nitrogen feeding regimes. Control of chlorophyll content ultimately gives cultivators greater ability to control the photosynthetic abilities of cultures without genetic manipulation.

Second-stage phototrophic growth utilizes the cultivated algal seed cell (P-07) product from the closed heterotrophic process. In a preferred embodiment, an open-air pond (E-5) or similar high surface area hybrid vessel wherein the algal culture is in full or partial contact with the external environment is utilized for growth. Spent broth from both the obligate and closed heterotrophic processes (P-06 and P-10) can be used as a nutritive source for the open-pond culture. The spent broth from the heterotrophic biomass production can either be concentrated via centrifuge or directly sent into the open pond together with the cultured seed cells without separation. Although, the phototrophic reactor volume is considerably larger than the heterotrophic process, the concentrated nutrients in the heterotrophic broths can serve as an important input to the ponds. Algae from the phototrophic growth ponds (P-14) are harvested (E-06). In a preferred embodiment of the downstream separation, the algal biomass (P-18) is directly converted to mixed fatty acid methyl esters (FAME) (P-21) and other by-products (P-19) using the esterification process that requires no oil extraction or drying (E-7) (PCT/US08/50799). However, other fuel conversion and fuel-upgrade methods, such as pyrolysis, liquefaction, gasification, etc. can be utilized from the recovered algal biomass (Li et al., 2008).

Co-location of the algal process near under-valued wastewater production facilities such as animal operations, industrial food processing facilities or municipal wastewater treatment plants can allow for an important added synergy.
Nutritive components can also be supplied by an associated anaerobic digestion (AD) unit that may employ a nutrient recovery system similar to that described in U.S. patent application Ser. No. 12/132,016, which is herein incorporated by reference. Wastewater, be it industrial, agricultural, manure, or municipal can be treated via AD to produce biogas which can be converted to combined-heat and power as well as an effluent that still contains the original quantity of nutrients as the influent minus some of the organic carbon. In this embodiment, the AD serves as a receiving vesicle for organic carbon wastes, producing heat and power that can in part be used in the algal cultivation process and nutritive-rich wastewater that can also be used in the algal process. In addition the phototrophic ponds will also require an inorganic carbon source in the form of CO₂ (P-13) which can be supplied in part from waste gas (P-09 and P-11) from the AD and associated generator (E-09) as well as from the waste gas from the heterotrophic seed reactors (P-05). Temperature control of the ponds can be in part controlled through use of the waste generator heat from the AD (P-23). Required electrical input can be drawn as a parasitic load to the electrical output from the AD generator (P-24). Concentrated cells harvested from the sedimentation tank (P-16) can then be processed in the aforementioned in situ extractor (E-07) to form biodiesel (P-22).
The parallel integration of the heterotrophic process with the phototrophic process will allow for the utilization of the waste organic carbon for algae biomass production in addition to the available resources (water, CO₂, sunlight), thus increasing the production capacity. Moreover, the heterotrophic and phototrophic processes can be alternatively operated singly or in parallel during low phototrophic algae productivity times such as cold climate winters or periods of contamination with organic carbon resources being utilized instead.
   To that end, the integrated system of the invention may provide one or more detectors (sensors) and/or controllers which sense conditions in one or more reactors and alert a user to conditions that are favorable or unfavorable for growth. For example, the phototrophic reactor may be equipped with a sensor that measures or senses the quantity or amount of sunlight or other light (e.g. in lumens) that impinges on the phototrophic culture at any given time, or the cumulative amount of sunlight that impinges on the phototrophic culture during a period of time of interest (e.g. during a minute, and hour, a day, etc.). The sensor may be designed so as to provide output to a user whereby a user can employ the output to decide whether to continue phototrophic culture, or to discontinue (stop, cease, shut down) phototrophic culture and switch to heterotrophic culture. Alternatively, the sensor may be designed to include or to transmit information to an automated controller that automatically switches the system to operate in heterotrophic rather than phototrophic mode, or vice versa (e.g. switch back to operating in both modes, i.e. start up phototrophic mode). In some embodiments, only the phototrophic reactor is so equipped. However, in other embodiments, the phototrophic reactor and/or the heterotrophic reactor is/are equipped to track conditions. Even though the heterotrophic reactor does not utilize sunlight, it may be situated in a manner that allows a sensor of relevant conditions to be associated therewith, and to record information and provide output that is utilized to cause an automatic switch away from or back to phototrophic mode, or a manual switch operated by a user to switch back to or away from phototrophic mode. Alternatively, such a detector may be electronically coupled or operably connected to both reactors.

In addition to sensing levels light, or instead of sensing light, the detector that is utilized (or additional detectors) may be designed to also monitor (track, sense, measure, etc.) other parameters, including but not limited to temperature, the presence of contaminants, the passage of time (e.g. a switch to fully heterotrophic mode may be may automatically in the fall, regardless of the amount of light or the temperature), or in response to conditions in one or both of the reactors (e.g. when the heterotrophic reactor is inoperable due to maintenance, malfunction, etc. the phototrophic reactor may also shut down), etc. Those of skill in the art will recognize that many useful parameters may be monitored, all of which are encompassed by the invention. Further, implementation of the automated switch to or away from a given mode may be accomplished by any means known the those of skill in the art, and may, for example, employ a computerized system which records input in terms of the parameter(s) being followed, and which can provide an output, e.g. information about the parameters, or instructions to a controller which can implement the switch between modes. Further, such detectors and controllers may be used to fine-tune the reactors, e.g. to detect and inform a user of the status (e.g. of seed production) in the heterotrophic reactor so as to “ramp up” (or down) the level of activity in the phototrophic reactor, or vice versa. In other words, modulation of the activity of the two reactors in response to sensors need not be all or nothing, but can be implemented by degrees, either manually or automatically. In fact, when multiple reactors are employed, the controllers may be equipped to determine how many of the reactors are operable at a given time, and which mode they carry out (phototrophic or heterotrophic). For example, during the summer season, a portion of the capacity of the heterotrophic process (e.g. 30%) is dedicated to seed production and a major portion (say 70%) for algae biomass production; when contamination occurs, part of the algae biomass production capacity will switch to seed production. During winter time, phototrophic production may stop altogether, and the portion for seed production will be switched and used for algae biomass production. Such arrangement will allow the efficient use of the facility and assure continuous algae biomass supply year round. This aspect of the invention is illustrated schematically in FIG. 1 as detector 10 coupled to optional automated controller 20.

In addition, in some embodiments of the invention, heterotrophic culture is also used for biomass and lipid accumulation. For example, in order to maximize reactor use, a heterotrophic reactor or culture that is no longer needed for seed production (or which has never been used for seed production) may be used for biomass and lipid accumulation e.g. in parallel with a heterotrophic/phototrophic process as described herein. Alternatively, if sufficient seed algae is produced (e.g. a level or amount that will suffice to meet the needs of the system for a desired period of time), then the heterotrophic reactor may simply be converted to use for biomass and lipid accumulation, at the same time as phototrophic biomass and lipid accumulation is occurring.

### Examples

### Example 1

### Flow Chart of Proposed System

FIG. 1 summarizes the incorporation of the heterotrophic and phototrophic growth process within an algal to biofuels production facility with Table 1 identifying key equipment and pipelines.

TABLE 1
Equipment and pipeline list for Figure 1

| | Description |
|---|---|
| Equipment List | |
| E-1 | Hydrolysis of waste organic carbon |
| E-2 | Heterotrophic algae or yeast culture |
| E-3 | Heterotrophic culture of seed cells for inoculation to phototrophic culture |
| E-5 | Open pond for phototrophic algae culture |
| E-6 | Algae biomass settler |
| E-7 | Wet algae oil extraction and separation |
| E-8 | Biodiesel production processor |
| E-9 | Electricity generator |
| E-10 | Detector |
| E-20 | Controller |
| Pipeline List | |
| P-01 | Waste organic carbon to hydrolysis |
| P-03 | Hydrolyzed waste organic carbon to seed cell culture tank |
| P-04 | Hydrolyzed waste organic carbon to heterotrophic culture |
| P-05 | Produced CO2 |
| P-07 | Heterotrophic cultured seed cells to open pond |
| P-10 | Residue N and P in effluent |
| P-11 | CO2 produced from generator |
| P-12 | Extra CO2 supplement |
| P-13 | CO2 to open pond |
| P-14 | phototrophic algae biomass in water |
| P-16 | Flocculated algae biomass slurry |
| P-17 | High value by-products from heterotrophic culture of algae or yeast |
| P-18 | Heterotrophic algae or yeast biomass |
| P-19 | Algae biomass residue after oil extraction |
| P-20 | Crude glycerol from biodiesel production |
| P-21 | Oil extracted from algae biomass |
| P-22 | Biodiesel produced |
| P-23 | Heat produced from generator can be used to control temperature |
| P-24 | Electricity produced from generator can be used to drive the equipment |

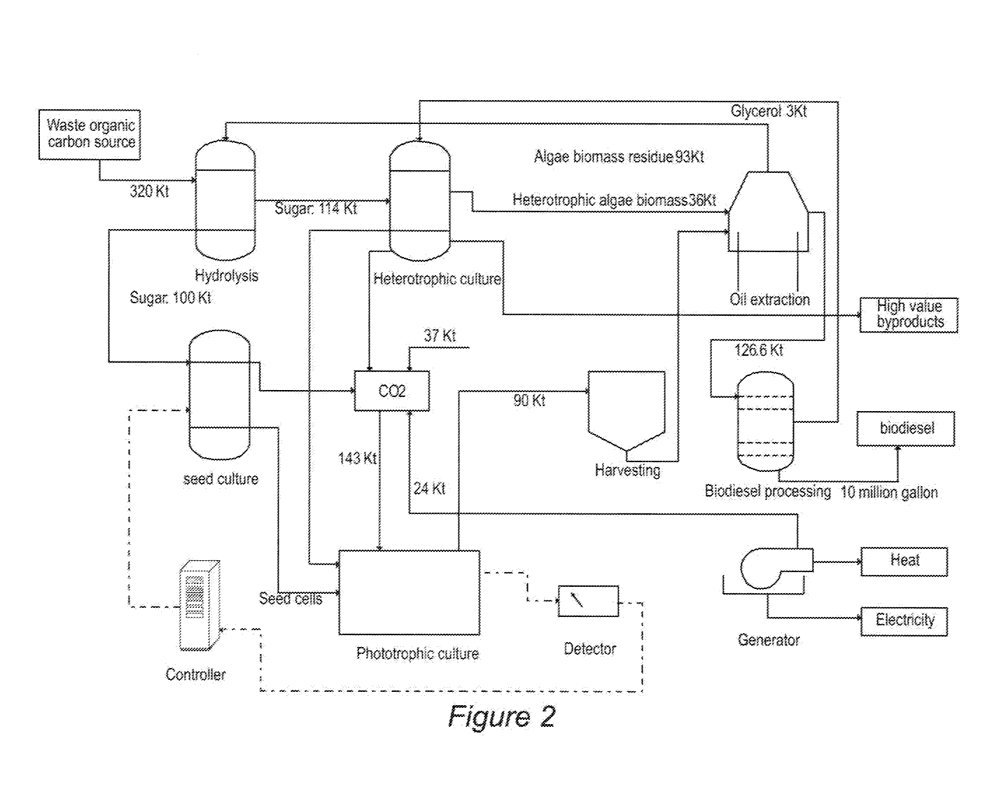

FIG. 2 is a mass balance for the integrated process, based on a 10 million gallon biodiesel production capacity and known yields for the individual processes as determined in laboratory and pilot-scale experiments. From the mass balance it can be noted that while making 10 MMg/yr of biodiesel via this process, all nutrient and inorganic carbon needs are for the most part met—requiring the need for only organic carbon input. Further analysis shows that ⅕ of the organic carbon input can be met with spent organic waste processed through the hydrolysis reactor or directly through the use of crude glycerol. Additional input needs in the form of a hydrolysis reactor and anaerobic digester can be in part offset by additional revenue streams including tipping fees, electricity and value-added products.

### Example 2

### Heterotrophic Growth Culture

Various organic waste streams can be used as feedstock for the heterotrophic culture process. After pre-treatment to various degrees, products will consist of sugars, small chain fatty acids and/or glycerols. Heterotrophic fermentation utilizing these varied carbon sources will be in large-scale fermentors with dedicated controlled of pH, dissolved oxygen, and temperature so as to provide an optimal condition for cell growth and cell density, which is known by the technical person familiar with state of the art. Most of the carbon and parts of the nitrogen and phosphorous will be consumed in this process however a certain amount of COD, nitrogen and phosphorous will remain in the effluent. Fortunately, the effluent will be used as a nutrient source for the phototrophic algae culture, allowing for cost reductions and greater recycling and utilization of system inputs. In turn, the effluent from the final phototrophic process will allow for even lower levels of COD, nitrogen, and phosphorus, as the phototrophic algae will both utilize and uptake them, thereby enhancing down-stream water quality. The examples provided within offer detailed information regarding: (1) the capability of algae and algae-like organisms to utilize waste organics in either a dedicated growth or seed-cell heterotrophic process and (2) the carbon, nitrogen and phosphorous available throughout the integrated process for various organic waste streams.

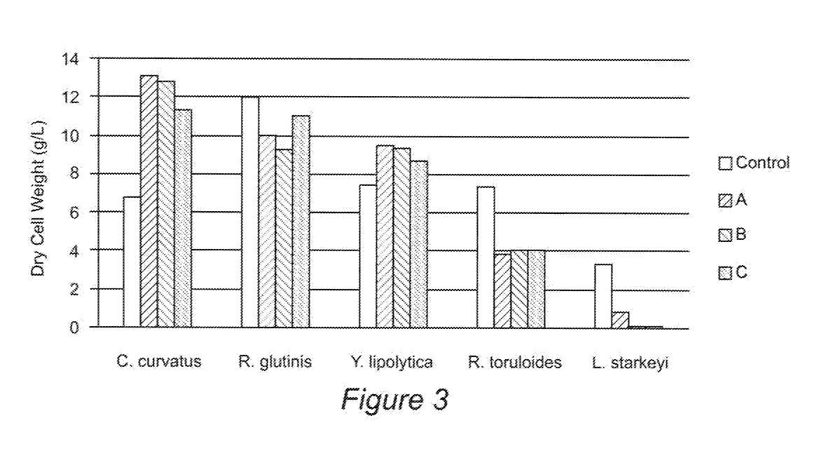

Oleaginous yeast culture with organic fraction municipal solid food scraps. Five species of oleaginous yeast, as shown in FIG. 3, are used as single cell oil producers for the heterotrophic production process (E2 in FIG. 1). The organic waste is used in this process as feedstock, and the food waste used as a typical example of organic waste. The liquid part of food waste hydrolyzed broth was used as the basic media (medium A), extra 5 g/L peptone and 5 g/L yeast extract were added for medium B, and further extra glucose (10 g/L) was added for medium C. Medium prepared with water was used as the control medium, which consisted of 5 g/L peptone, 5 g/L yeast extract, and 20 g/L glucose. Each species was cultured with these four media treatments to test growth potential in the medium prepared with food waste hydrolyzed broth. With food waste hydrolyzed broth alone, the oleaginous yeast Rhodotorula glutinis produced 10 g/L biomass, and Cryptococcus curvatus and Yarrowia lipolytica produced more biomass than the control medium (FIG. 3), showing that the selected oleaginous yeast, algae and algae-like species have strong capabilities in regard to high-density culture utilizing waste organic resources.

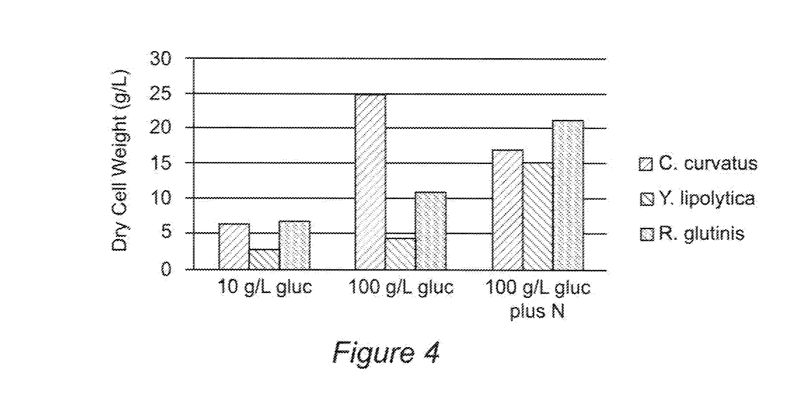

Oleaginous yeast cultured in municipal wastewater. The data shows that if sufficient carbon and nitrogen sources are provided, all three of the selected oleaginous yeast species, and presumably other algae and algae-like species, would have good growth (>15 g/L) in the municipal wastewater environment (FIG. 4), showing that municipal wastewater alongside the presence of additional sources of carbon and nitrogen can serve as a potential media for yeast, algae and algae-like biomass development.

A two-step process for culture of oleaginous yeast using food waste and municipal wastewater. Table 2 shows the main components of hydrolyzed food waste used in this culture.

TABLE 2
Composition of the hydrolyzed food waste

| Components | g/L | % of total sugar |
|---|---|---|
| Arabinose | 0.8 ± 0.2 | 1.6 ± 0.5% |
| Galactose | 1.3 ± 0.3 | 2.6 ± 0.1% |
| Glucose | 47.4 ± 9.6 | 92.4 ± 1.1% |
| Mann/Xylo | 1.0 ± 0.2 | 2.0 ± 0.4% |
| Fructose | 0.8 ± 0.3 | 1.5 ± 0.8% |
| total sugar | 51.2 ± 9.7 | n/a |
| Total COD (g/L) | 106.3 ± 3.3 | n/a |
| Total nitrogen (g/L) | 1.9 ± 0.8 | n/a |
| Total phosphorus (g/L of PO₄) | 1.8 ± 0.2 | n/a |

The hydrolyzed food waste was mixed with municipal wastewater, and used as the culture media for the oleaginous yeast. The first-step culture lasted 6 days, with most of the nutrients and the oleaginous yeast biomass being produced in this step. After this step, 90% of the produced yeast biomass was harvested and 10% of the yeast biomass was kept in the effluent to have further growth and nutrient sequestration. Further biomass was produced in the 2^{nd} step culture, with a lower efficiency than the first step, but the concentration of COD, nitrogen and phosphorous were decreased to a much lower level. The effluent from this process was then used to support phototrophic growth of C. sorokiniana, since there was still a good amount of nitrogen and phosphorous available for use by the algae. Phototrophic algae biomass was produced in this step, and the concentration of COD, nitrogen and phosphorous were decreased to a level that can be discharged without environment pollution (Table 3).

TABLE 3
Two-step yeast culture process with food waste and municipal wastewater

| | | Cryptococcus curvatus | Rhodotorula glutinis |
|---|---|---|---|
| 1^{st} step yeast culture | Biomass dry weight (g/L) | 7.5 ± 0.3 | 5.2 ± 1.2 |
| | COD (g/L) | 3.8 ± 0.6 | 8.3 ± 1.2 |
| | N (g/L) | 0.42 ± 0.10 | 0.54 ± 0.03 |
| | P (mg/L of PO₄⁻) | 22 ± 2 | 25 ± 5 |
| | TFA/Biomass (%) | 28.6 ± 2.2% | 19.6 ± 0.2% |
| 2^{nd} step yeast culture | Biomass dry weight (g/L) | 1.1 ± 0.1 | 1.5 ± 0.3 |
| | COD (g/L) | 2.5 ± 0.2 | 3.2 ± 0.2 |
| | N (g/L) | 0.19 ± 0.07 | 0.14 ± 0.01 |
| | P (mg/L of PO₄⁻) | 19 ± 6 | 14 ± 7 |
| | TFA/Biomass (%) | 20.0 ± 2.5% | 18.7 ± 9.0% |
| Phototrophic | Biomass dry weight (g/L) | 1.53 | 0.58 |
| culture of C. | COD (mg/L) | 1033 ± 58 | 1200 ± 173 |
| sorokiniana | N (mg/L) | 33 ± 4 | 34 ± 6 |
| | P (mg/L of PO₄⁻) | 15 ± 5 | 6 ± 2 |
| | TFA/Biomass (%) | 22.9 ± 2.4% | 20.0 ± 0.7% |

Heterotrophic seed cell culture of C. sorokiniana using food waste and municipal wastewater. Food waste and wastewater media were used to culture the seed cells of C. sorokiniana. With a 6 day heterotrophic culture, C. sorokiniana grew to 353 and 366 million cells/mL, and the nutrients were consumed in this process (Table 4).

TABLE 4
Heterotrophic seed cells of C. sorokiniana
with food waste and wastewater

| | High carbon | Low carbon |
|---|---|---|
| Biomass dry weight (g/L) | 4.6 ± 0.8 | 3.2 ± 0.2 |
| Cell density (million cells/mL) | 353 ± 84 | 366 ± 52 |
| COD (g/L) | 8.5 ± 1.4 | 3.9 ± 0.4 |
| N (g/L) | 0.79 ± 0.32 | 0.19 ± 0.03 |
| P (mg/L of PO₄⁻) | 179 ± 49 | 65 ± 9 |
| TFA/Biomass (%) | 26.5 ± 3.0% | 20.0 ± 0.6% |

Then, the heterotrophic cultured seed cells, together with the culture medium, was inoculated to phototrophic culture with an inoculate rate of 1% and 0.1%. With another 7 days phototrophic culture, 0.16 g/L and 0.15 g/L algae biomass was produced respectively (Table 5).

TABLE 5
The phototrophic growth of seed cells of C. sorokiniana
from food waste culture

| | 1% inoculate | 0.1% inoculate |
|---|---|---|
| Biomass dry weight (g/L) | 0.16 ± 0.01 | 0.15 ± 0.02 |
| TFA/Biomass (%) | 15.2 ± 3.0% | 14.3 ± 0.5% |

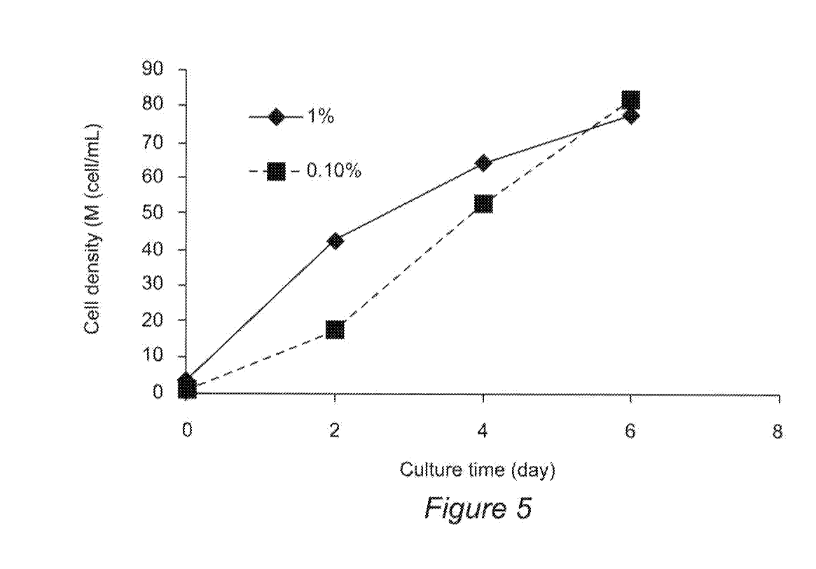

The growth kinetics of the C. sorokiniana is shown in FIG. 5. This data shows that not only can C. sorokiniana, an example dual-trophic organism, grow effectively on food waste in a heterotrophic seed cell process, but the resulting seed cells were capable of under-going trophic conversion and growing effectively in a phototrophic process.

### Example 3

### Other Available Algae Species can be Used in this Process

Besides Chlorella sorokiniana, as shown in examples 2, 4, and 5, a variety of other algae species can be cultured at both heterotrophic and phototrophic culture conditions and can be used in this process as described in examples 1 and 2. Although no experimental data on cultivation of these species within this specific process is shown here, it is obvious that these species can be used as production microorganism species, since it has been proved the ability of both phototrophic and heterotrophic growth

TABLE 6
Examples of algae that can grow under
heterotrophic and/or phototrophic conditions

| Available species | References |
|---|---|
| Chlorella sorokiniana | Chen & Johns 1991 |
| Chlorella vulgaris | Karlander & Krauss 1965 |
| Chlorella Kessleri | Rezenka et al., 1983 |
| Chlorella protothecoids | Li et al., 2007 |
| Chlorella pyrenoidosa | Theriault 1964 |
| Tetraselmis suecica | Jo et al., 2004 |
| Scenedesmus obliquus | Abeliovich & Weisman 1978 |
| Scenedesmus acutus | Sandmann & Boger 1981 |
| Chlamydomonas reinhardtii | Graves et al., 2008 |
| Chlamydomonas dysosmos | Lewin 1954 |
| Nitzschia closterium | Lewin 1958 |
| Euglena gracilis | Peak & Peak 2007 |
| Navicula pellieubsa | Lewin 1953 |
| Nitzschia obtusa | Lewin 1953 |

### Example 4

### Growth Rate Comparisons of Heterotrophic and Phototrophic Seed Culture

A key concept within the invention is the ability of the heterotrophic process to serve as a superior seed process than the commonly used phototrophic seeding process, prior to entry into the main phototrophic growth pond. Algae C. sorokiniana (UTEX 1602), as an example organism for the proposed process, was aseptically inoculated into 250 ml Erlenmeyer flasks at the same inoculum rate and cultured in Kuhl medium, under separate phototrophic and heterotrophic conditions, as shown in Table 7.

TABLE 7
Kuhl medium composition

| Ingredients | Unit | Phototrophic | Heterotrophic |
|---|---|---|---|
| Glucose | g/L | — | 10 |
| KNO₃ | mg/L | 1011.1 | 1011.1 |
| NaH₂PO₄•H₂0 | mg/L | 621 | 621 |
| Na₂HPO₄•2H₂0 | mg/L | 89 | 89 |
| MgSO₄•7H₂0 | mg/L | 246.5 | 246.5 |
| EDTA | mg/L | 9.3 | 9.3 |
| H₃BO₃ | mg/L | 0.061 | 0.061 |
| CaCl₂•2H₂0 | mg/L | 14.7 | 14.7 |
| FeSO₄•7H₂0 | mg/L | 6.95 | 6.95 |
| ZnSO₄•7H₂O | mg/L | 0.287 | 0.287 |
| MnSO₄•H₂0 | mg/L | 0.169 | 0.169 |
| (NH₄)₆Mo₇O₂₄•4H₂O | mg/L | 0.01235 | 0.01235 |
| CuSO₄•5H₂0 | mg/L | 0.00249 | 0.00249 |
| pH | — | 6.1 | 6.1 |

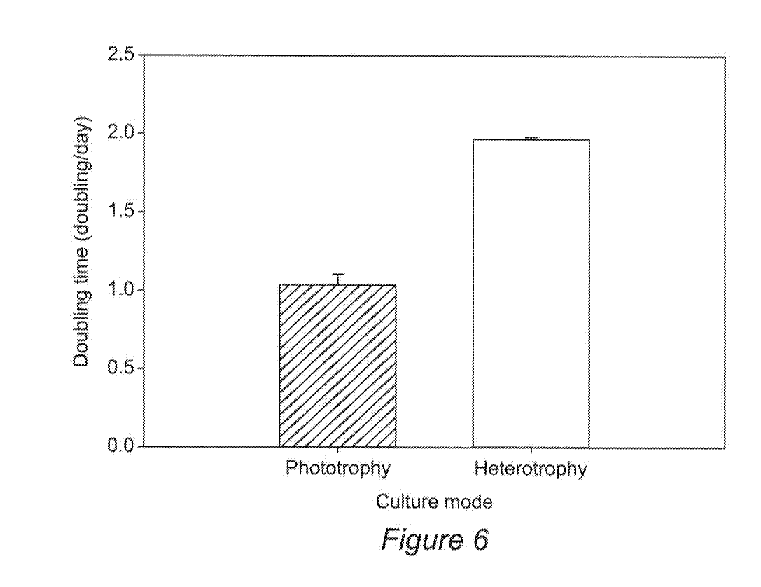

The heterotrophic seed culture was conducted at 27° C. in the dark, while the phototrophic seed culture was conducted with continuous CO₂ bubbling at 27° C. under light. After 6 days culture, the growth rates were compared as shown in FIG. 6. The doubling time of phototrophic seed culture was 1.0 doubling per day, while the heterotrophic culture was 2.0 doublings per day. This result indicates that the efficiency of the heterotrophic process was twice that of the phototrophic process in regard to producing seed cells. This heterotrophic advantage should only be enhanced upon phototrophic scale-up (light limitation) as well as large-scale use of a fermentor (increased capabilities in regard to aeration and agitation).

### Example 5

### Contamination Control through Heterotrophic Seed Culture—Lab Culture

Open ponds are low-cost systems for large-scale algae cultivation but it is often difficult to maintain mono-specific algal cultures within them. Contaminants, such as undesired algal species and bacteria, may enter an open-pond algal system at a very low concentration, but can compete for nutrients and other resources, ultimately destroying the monoculture system with superior growth rates. A contamination control method for keeping the dominance of a particular algal species in an open system is to accelerate their growth and harvest before other undesired species can grow to a deleterious density. In some embodiments, increasing desired algal inoculum size can shorten the lag phase so that the algae can start their exponential phase earlier and reach the stationary phase more rapidly before the foreign species attain a harmful density. In such embodiments, the heterotrophic culture system described above can provide sufficient and high-density seed for this contamination control strategy. An experimental set-up was devised to determine the extent to which heterotrophic seed culture and its development of high-density and effectively-sized cells can provide enhanced protection against contamination.

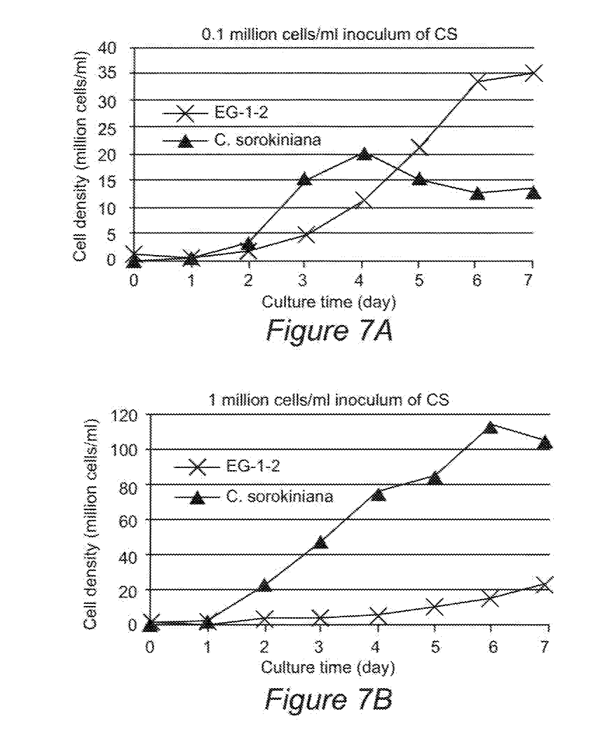

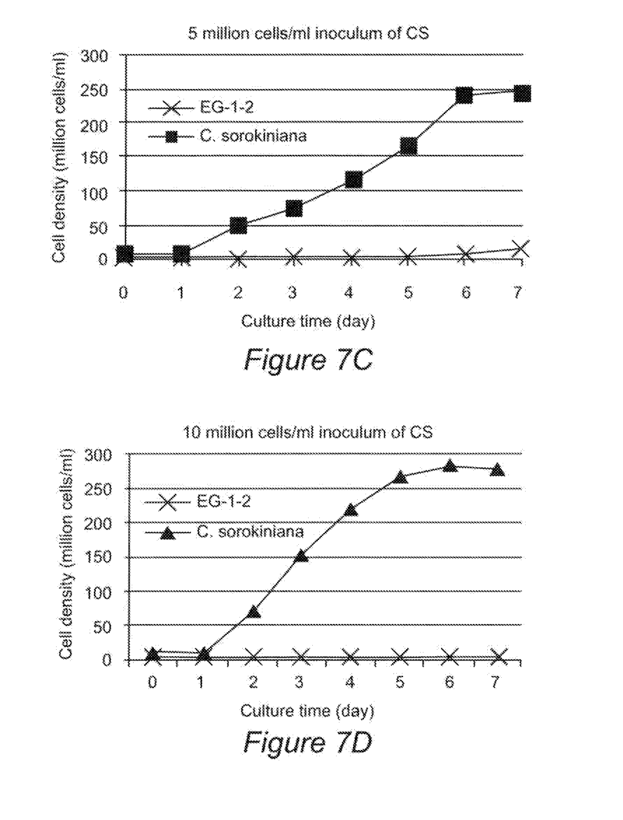

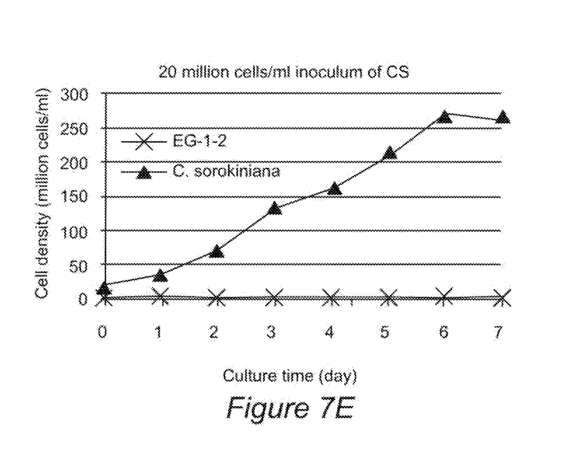

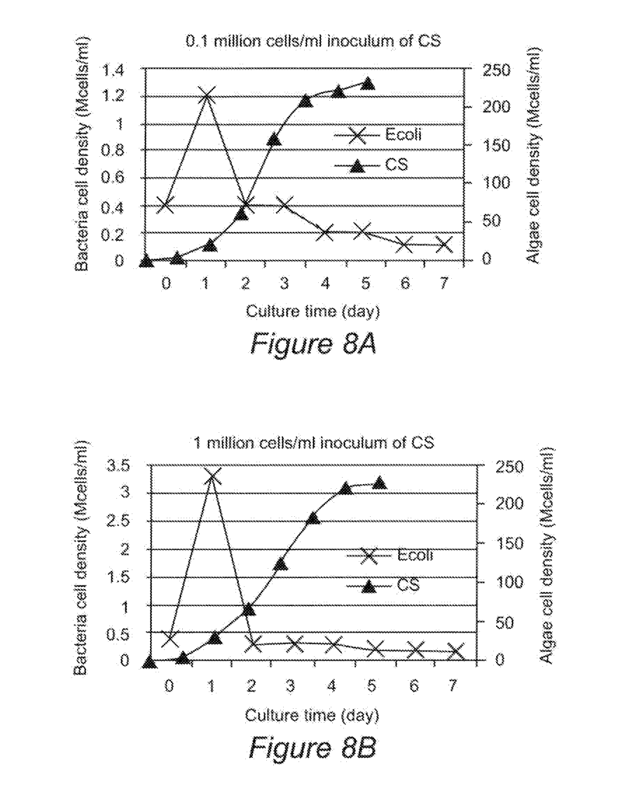

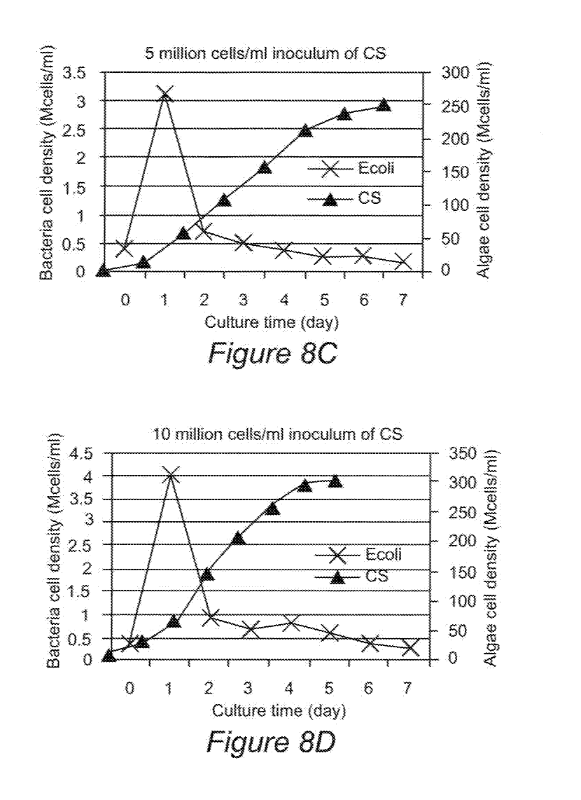

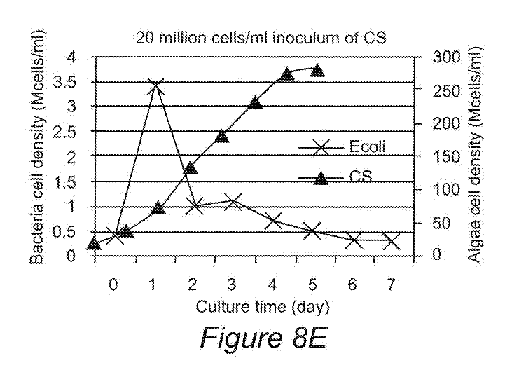

C. sorokiniana (CS) (UTEX 1602) was used as the desired algae species while native algae species in the Pacific Northwest, EG-1-2 and E. coli, were used as the contaminants. CS and EG-1-2 were inoculated into the same Erlenmeyer flask for phototrophic culture. The inoculum size for EG-1-2 was fixed at 1 million cells/ml while CS was inoculated with different inoculum sizes. The same experiment design was used for CS and E. coli. The results are shown in FIGS. 7-8. As shown in FIG. 7, EG-1-2 was dominant when the inoculum size for CS was only 0.1 million cells/ml, however as inoculums size of CS increased, EG-1-2 gradually lost its dominant position. These results indicate that by increasing the inoculum size of the desired algae species contamination by other foreign algae could be controlled, and more specifically that use of heterotrophic seeding as a means to intensify inoculums size can be beneficial in controlling contamination. The bacteria E. coli was introduced as a contaminant because the heterotrophic cultured algae seeds would bring some organic carbon with them when they were inoculated into the open-pond system. As shown in FIG. 8, E. coli could grow a little bit when the inoculum size of CS was only 0.1 million cells/ml (brought the lowest organic carbon). With increasing inoculum size of CS, the cell density of E. coli increased, however, E. coli could only keep growing at the first day, after that its cell density dropped to a very low level. And also E. coli did not have significant negative impact on the growth of CS no matter how much the inoculum size. These results also support the large inoculum size and heterotophic seeding strategy described above.

### Example 6

### Growth Rate and Productivity of the Integrated Systems

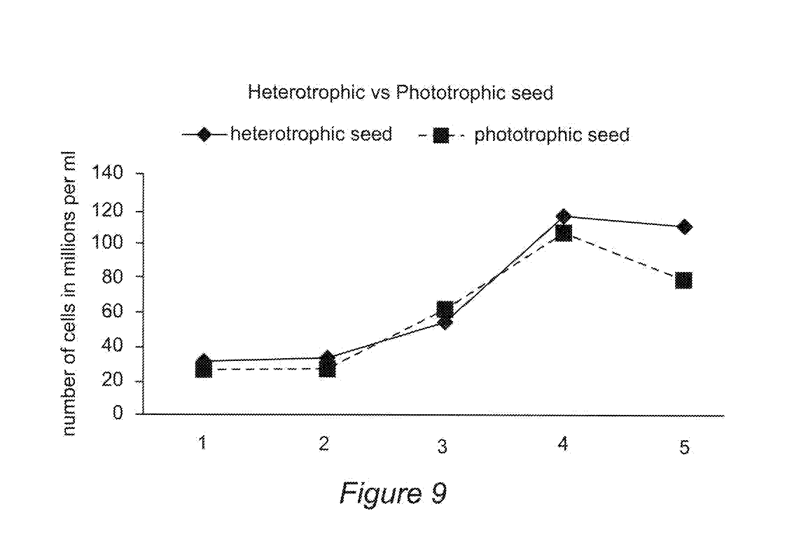

Seed cells for large-scale algae cultivation are of utmost importance for successful growth systems. One main objective for an integrated system is to reduce cost associated with individual systems by using waste and products from one system with another system. In this invention, use of the heterotrophic seed operation using waste organics from other systems allows not only for enhanced inoculums and greater protection against contamination, but importantly results in lower operational costs while still maintaining comparable yields and productivities in regard to end product development. Here we show that algae grown heterotrophically or phototrophically as seeds to inoculate ˜50 liter open cultures and have comparable growth rates as shown in FIG. 9.

### Example 7

### Cost Comparison of Mixed-Trophic and Phototrophic-Alone Systems for Algal Culture

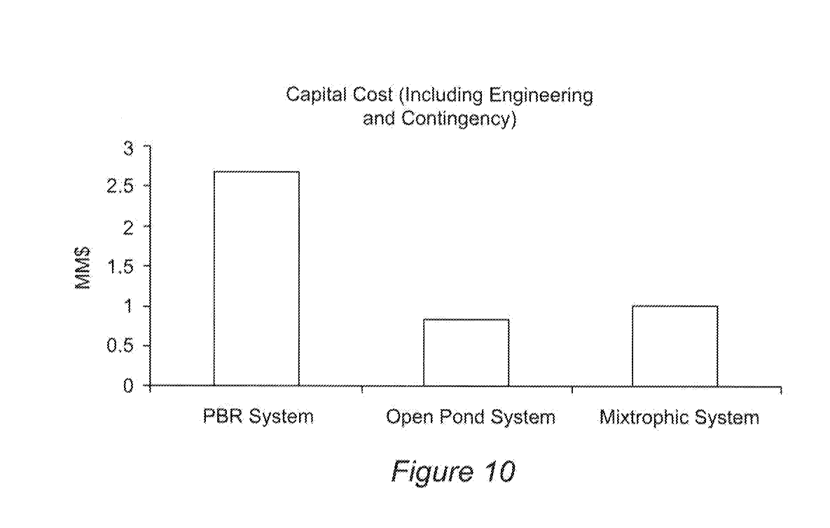

Computer modeling can greatly aid industry in analyzing theoretical yields versus actual yields, cultivation practices, harvest efficiency, extraction efficiencies, nutrient cycling, watershed practices, costs, and more. In this example, economic assessment of two culture systems (mixed-trophic and phototrophic-alone) has been made on the basis of known reference data in regard to equipment capital costs, operating expenses, productivities, etc. The calculation was based upon a facility with 24 open-ponds each with an area of 80,000 m² and a depth of 0.15 m. Final analysis as described in FIG. 10, shows that in comparison, the two systems have essentially equal investment costs, although their equipment and indirect costs are slightly different.

While the invention has been described with reference to certain preferred examples, those of skill in the art will recognize that the invention can be practiced with modification within the scope of the appended claims.

### REFERENCES

Abeliovich, A., and Weisman, D. (1978). ROLE OF HETEROTROPHIC NUTRITION IN GROWTH OF ALGA SCENEDESMUS-OBLIQUUS IN HIGH-RATE OXIDATION PONDS. Applied and Environmental Microbiology 35, 32-37.
Bailey, R. B., DiMasi, D., Hansen, J. M., Mirrasoul, P. J., Ruecker, C. M., Veeder, G. T., III, Kaneko, T., and Barclay, W. R. (2003). Enhanced production of lipids containing polyenoic fatty acid by very high density cultures of eukaryotic microbes in fermentors. U.S. Pat. No. 6,607,900.
Boyle, N. R., and Morgan, J. A. (2009). Flux balance analysis of primary metabolism in Chlamydomonas reinhardtii. Bmc Systems Biology 3.
Chen, F., and Johns, M. R. (1991). EFFECT OF CARBON TO NITROGEN RATIO AND AERATION ON THE FATTY ACID COMPOSITION OF HETEROTROPHIC CHLORELLA-SOROKINIANA. Journal of Applied Phycology 3, 203-210.
Chen, G. Q., and Chen, F. (2006). Growing phototrophic cells without light. Biotechnology Letters 28, 607-616.
Chisti, Y. (2007). Biodiesel from microalgae. Biotechnology Advances 25, 294-306.
Easterling, E. R., French, W. T., Hernandez, R., and Licha, M. (2009). The effect of glycerol as a sole and secondary substrate on the growth and fatty acid composition of Rhodotorula glutinis. Bioresource Technology 100, 356-361.
Graves, D. A., Spradlin, G. M., Greenbaum, E. (2008). Effect of oxygen on photo auxotrophic and heterotrophic growth of Chlamydomonas reinhardtii in an anoxic atmosphere. Photochemistry and photobiology, 52, 585-590.
Hermsmeier, D., Mala, E., Schulz, R., Thielmann, J., Galland, P., and Senger, H. (1991).

ANTAGONISTIC BLUE-LIGHT AND RED-LIGHT REGULATION OF CAB-GENE EXPRESSION DURING PHOTOSYNTHETIC ADAPTATION IN SCENEDESMUS-OBLIQUUS. Journal of Photochemistry and Photobiology B-Biology 11, 189-202.
Jo, Q., Choy, E. J., Park, D. W., and Veron, B. (2004). Sterol dynamics of heterotrophic Tetraselmis suecica and its nutritional implication in the bivalve aquaculture. Aquaculture Research. 35, 371-377

Karlander, E. P. and Krauss, R. W. (1966) Responses of Heterotrophic Cultures of Chlorella vulgaris Beyerinck to Darkness and Light. I. Pigment and pH Changes, Plant Physiology, 41, 1-6
Kapdan, I. K., and Kargi, F. (2006). Bio-hydrogen production from waste materials. Enzyme and Microbial Technology 38, 569-582.
Knuckey, R. M., Brown, M. R., Robert, R., and Frampton, D. M. F. (2006). Production of microalgal concentrates by flocculation and their assessment as aquaculture feeds. Aquacultural Engineering 35, 300-313.
Kyle, D., and Ratledge, C. (1992). industrial applications of signle cell oils.
Lewin, J. C. (1953). Heterotrophy in Diatoms. Journal of General Microbiology 9 305-313.
Lewin, R. A. (1954) Utilization of acetate by wild type and mutant Chlamydomonas dysosmos. Journal of General Microbiology. 11, 459-471.
Lewin, J. C. (1958). The taxonomic position of phaedactylum tricornutum. Journal of General Microbiology. 18, 427-432.
Li, Y., Horsman, M., Wu, N., Lan, C. Q., and Dubois-Calero, N. (2008). Biofuels from microalgae. Biotechnology Progress 24, 815-820.
Li, X., Xu, H., Wu, Q., (2007) Large-scale biodiesel production from microalga Chlorella protothecoides through heterotrophic cultivation in bioreactors, Biotechnology and Bioengineering, 98, 764-791
Oyler, J. R. (2008). TWO-STAGE PROCESS FOR PRODUCING OIL FROM MICROALGAE. In “us patent, 20080160593”.
Peak, J. G. and Peak, M. J. (2007). Heterotrophic (Dark) CO₂ Fixation by Euglena gracilis. Possible regulation by tricarboxylic acid cycle intermediates. Journal of Eukaryotic Microbiology. 23, 165-167.
Pienkos, P. T., and Darzins, Al (2009). The promise and challenges of microalgal-derived biofuels. Biofuels, Bioproducts and Biorefining 3, 431-440.
Rezanka, T., Prikrylova, V., Yarotsky, S. V., and M. Podojil, (1983) Monosaccharides of the green fresh-water alga Chlorella kessleri, 28, 297-291
Rusch, K. A., and Christensen, M. J. (1998). Description and operation of a hydraulically integrated serial turbidostat algal reactor (HISTAR). Journal of Shellfish Research 17, 338.
Rusch, K. A., and Malone, R. F. (1998). Microalgal production using a hydraulically integrated serial turbidostat algal reactor (HISTAR): a conceptual model. Aquacultural Engineering 18, 251-264.
Sandmann, G and Boger, P. (1981) Influence of light on plastocyanin formation in the alga Scenedesmus aeutus. Photosynthesis Research, 2, 281-289.
Singh, A., and Ward, O. P. (1997). Microbial production of docosahexaenoic acid (DHA, C22:6). Advances in Applied Microbiology, Vol 45 45, 271-312.
Theegala, C. S., Malone, R. F., and Rusch, K. A. (1999). Contaminant washout in a hydraulically integrated serial turbidostat algal reactor (HISTAR). Aquacultural Engineering 19, 223-241.

Theriault, R. J. (1965) Heterotrophic Growth and Production of Xanthophylls by Chlorella pyrenoidosa, Applied Microbiology, 13(3): 402-416.
Ward, O. P., and Singh, A. (2005). Omega-3/6 fatty acids: Alternative sources of production. Process Biochemistry 40, 3627-3652.
Wen, Z. Y., and Chen, F. (2003). Heterotrophic production of eicosapentaenoic acid by microalgae. Biotechnology Advances 21, 273-294.
Xiong, W., Li, X. F., Xiang, J. Y., and Wu, Q. Y. (2008). High-density fermentation of microalga Chlorella protothecoides in bioreactor for microbio-diesel production. Applied Microbiology and Biotechnology 78, 29-36.
Xue, F. Y., Miao, J. X., Zhang, X., Luo, H., and Tan, T. W. (2008). Studies on lipid production by Rhodotorula glutinis fermentation using monosodium glutamate wastewater as culture medium. Bioresource Technology 99, 5923-5927.

1. A method of producing biofuel feedstock, comprising the steps of:
using heterotrophic culture of algae or algae like species for seed production, said algae or algae like species having physiological mechanisms for both phototrophic and heterotrophic growth; and then
using seed produced from said heterotrophic culture in a phototrophic culture for accumulation of biomass by said algae or algae like species.

2. The method of claim 1, wherein said biomass includes lipids.

3. The method of claim 1 wherein said using heterotrophic culture step includes the step of using waste material as an organic carbon source, to culture said algae or algae like species having physiological mechanisms for both phototrophic and heterotrophic growth in a dark fermenter or bioreactor.

4. The method of claim 3, wherein said waste material is selected from waste water and waste solids.

5. The method of claim 4 wherein said waste water and waste solids are selected from the group consisting of agricultural wastes, industrial pulps, organic fraction municipal waste, and algae biomass residue, and any other organic waste which contains available organic carbon to be utilized by the cultured microorganisms.

6. The method of claim 1 further comprising the step of inputting carbon dioxide produced during said using heterotrophic culture step as a carbon source into said using phototropic culture step.

7. The method of claim 1 wherein said algae or algae like species are selected from the group consisting of Chlorella sp., Chlamydomonas sp., and Scenedesmus sp.

8. The method of claim 1 wherein said seed produced from said heterotrophic culture has a cell count ranging from 10⁷ to 10⁹ cells/ml, or more.

9. The method of claim 1, further comprising the step of using heterotrophic culture for accumulation of biomass by said algae or algae like species.

10. A method of producing biofuel, comprising the steps of:
using heterotrophic culture of algae or algae like species for seed production, said algae or algae like species having physiological mechanisms for both phototrophic and heterotrophic growth; and then
using seed produced from said heterotrophic culture in a phototrophic culture for biomass and lipid accumulation in said algae or algae like species; and
recovering lipids from algae or algae like species for use as biofuel.

11. The method of claim 10 wherein said using heterotrophic culture step includes the step of using waste material as an organic carbon source, to culture said algae or algae like species having physiological mechanisms for both phototrophic and heterotrophic growth in a dark fermenter or bioreactor.

12. The method of claim 11, wherein said waste material is selected from waste water and waste solids.

13. The method of claim 12 wherein said waste water and waste solids are selected from the group consisting of agricultural wastes, industrial pulps, organic fraction municipal waste, and algae biomass residue.

14. The method of claim 10 further comprising the step of inputting carbon dioxide produced during said using heterotrophic culture step as a carbon source into said using phototropic culture step.

15. The method of claim 10 wherein said algae or algae like species are selected from the group consisting of Chlorella sp., Chlamydomonas sp., and Scenedesmus sp.

16. The method of claim 10 wherein said seed produced from said heterotrophic culture has a cell count ranging from 10⁷ to 10⁹ cells/ml, or more.

17. A system for producing biofuel feedstock, comprising:
at least one tank for heterotrophic culture of algae or algae like species for seed production, said algae or algae like species having physiological mechanisms for both phototrophic and heterotrophic growth; and
at least one open pond or reactor for phototrophic culture for accumulation of biomass by said algae or algae like species using seed produced from said heterotrophic culture, said at least one open pond or reactor being configured so as to receive seed algae or algae like species from said at least one tank for heterotrophic culture of algae or algae like species for seed production.

18. The system of claim 17, further comprising at least one tank for heterotrophic culture of algae or algae like species for biomass accumulation by said algae or algae like species.

19. The system of claim 17, wherein said biomass includes lipids.

20. The system of claim 17, further comprising one or more detectors to detect and provide output regarding growth conditions in said at least one open pond or reactor.

21. The system of claim 20, further comprising one or more controllers to automatically shut down or start up operation of said at least one open pond or reactor in response to said output provided by said one or more detectors.

22. The system of claim 20, wherein said growth conditions are selected from amount of light, temperature, and presence of contamination.
